(19) United States
(12) Patent Application Publication
(10) Pub. No.: US 2024/0175816 A1
(43) Pub. Date: May 30, 2024

(54) METHOD OF DETECTION

(71) Applicant: THE UNIVERSITY OF QUEENSLAND, St. Lucia, Queensland (AU)

(72) Inventors: Paul Leslie BURN, Kenmore (AU); Paul Edward SHAW, Chelmer (AU); Shengqiang FAN, Chapel Hill (AU)

(21) Appl. No.: 18/552,340

(22) PCT Filed: Mar. 26, 2021
(86) PCT No.: PCT/AU2021/050271

§ 371 Date: Sep. 25, 2023

(51) Int. Cl. G01N 21/64; C07D 285/14

(52) U.S. Cl. CPC G01N 21/6428; C07D 285/14; G01N 2021/6432

(57) ABSTRACT

A method for detection of a nerve agent in a sample, which method comprises (a) irradiating an optical sensing element, the optical sensing element comprising a fluorescent sensing compound provided on a substrate, and measuring the luminescence of the optical sensing element; wherein the fluorescent sensing compound comprises a combination of at least one electron acceptor moiety and, optionally, one or more electron donor moieties such that the electronic properties of the sensing compound are sufficient to enable a change in the luminescence of the sensing element in the presence of the nerve agent, and a moiety that influences solubility of the sensing compound in a solvent; (b) contacting the sample with the optical sensing element; (c) measuring the luminescence of the optical sensing element; and (d) determining whether the nerve agent is present in the sample based on the measurements obtained in steps (a) and (c).

### FIELD OF THE INVENTION

This invention relates generally to the detection of nerve agents, particularly V-series nerve agents. In particular, the present invention relates to methods of detecting nerve agents using an optical sensing element. Specificity for detection of V-series nerve agents over G-series nerve agents is also described.

### BACKGROUND OF THE INVENTION

There is an on-going need to develop techniques for detection of chemical warfare agents (CWAs). In particular, there is a need for detection methods that are rapid or sensitive and have capability to detect agents at low concentrations. There is also a desire for detection methods that are selective and capable of differentiating between different classes of chemical warfare agents.

CWAs are classified into several groups, including nerve, blister, blood, choking, harassing, and incapacitation agents and toxins. In general, they were initially developed for military purposes but, given the relative ease of synthesis, they are available to terrorist groups and pose a real threat to public security. Nerve agents comprise a family of highly toxic organophosphate (OP) compounds [S. Costanzi, J.-H. Machado, M. Mitchell, ACS Chem. Neurosci. 2018, 9, 873].

Nerve agents generally enter the body through inhalation or via the skin and lead to deleterious effects on human health through interference with nerve function. Nerve agents act by inhibiting the enzyme acetylcholinesterase (AChE), which is critical for hydrolysing the neurotransmitter acetylcholine to control its concentration in the body [R. T. Delfino et al., J. Braz. Chem. Soc. 2009, 20, 407]. The inhibition of AChE can cause an accumulation of acetylcholine and result in muscle overstimulation.

Nerve agents are generally divided into two families, G and V. G-agents, including tabun (GA), sarin (GB), soman (GD) and cyclosarin (GF), were first developed before and during World War II. V-agents were synthesised later in the 1950s. A third family of nerve agents is referred to as Novichok but their molecular structures have not yet been confirmed [T. C. C. Franca et al., Int. J. Mol. Sci. 2019, 20,1222; Mirzayanov, V.S. State Secrets: An Insider's Chronicle of the Russian Chemical Weapons Program; Outskirts Press, Inc.: Parker, CO, USA, 2009; ISBN 1432725661].

Compared to G-series agents, V-series agents are more persistent in the environment due to them being less volatile and slower to hydrolyse. In addition, the V-agents are more toxic, with LD₅₀ (lethal dose, 50%) values of one to two orders of magnitude lower than the G-series agents [S. W. Wiener et al., J. Intensive Care Med. 2004, 19, 22]. In view of this, in the case of a battle field or terrorist attack scene, detection of the presence of a V-series agent is critical to allow implementation of appropriate protective measures and medical treatment.

Existing methods for the detection of V-series nerve agents include gas chromatography (GC), liquid chromatography (LC), ion mobility spectrometry (IMS), and Fourier transform infrared spectrometry (FTIR) [F. N. Diauudin et al., Sensing and Bio-Sensing Research, 2019, 26, 100305].

These instrument based techniques are stable and sensitive but they require expensive and bulky instrumentation and have substantial power requirements. In addition, the analytical techniques and sample preparation are complex and the instrumentation requires operation by skilled personnel; therefore these techniques do not meet the requirements for incident detection in the field.

Biosensing using enzymes, antibodies, or aptamers is an appropriate approach for on-site detection but it is limited by sensitivity or poor stability of the enzyme with respect to temperature and pH. In addition, the technique is expensive due to high enzyme production cost and difficulty for mass production [F. N. Diauudin et al. Sensing and Bio-Sensing Research, 2019, 26, 100305].

Colourimetric detection of nerve agents is another possible approach for on-site detection but this technique has a drawback of the requirement for liquid sampling in the form of liquid droplets. The technique relies on a chemical reaction, usually resulting in displacement of a substituent such as cyano, chloro or fluoro from the P atom. The adduct formed in the reaction is then detected. Since the technique is based on a chemical reaction, this means that the sensing element cannot be reused [V. Pitschmann et al., Chemosensors 2019, 7, 30]. This lack of reversibility results in limited applicability of the technique.

Fluorescence-based detection methodology has been reported using chemical reactions or coordination mechanisms, however the sensing material has been used in solution phase for the detection of V-series agents [X. Sun et al., 2017, 129, 9650; G. H. Dennison et al., Chem. Commun. 2014, 50, 195; G. H. Dennison et al., RSC Adv. 2014, 4, 55524] or loaded on a filter paper to detect nerve agent droplets [G. H. Dennison et al., RSC Adv. 2019, 9, 7615; A. J. Metherell, et al., J. Mater. Chem. C 2016, 4, 9664].

A key problem with fluorescence-based detection of nerve agents using a chemical reaction with the sensing material is that the nerve agents themselves are not particularly stable. [S. Fan et al., “Challenges in fluorescence detection of chemical warfare agent vapors using solid-state films”, Adv. Mater., 2019, 1905785]. As a consequence, the acid breakdown products can often interact with the sensing material in a similar manner as the nerve agent or other commonly available acids, e.g., acetic acid. In addition, the chemical reaction is irreversible meaning the sensor cannot be reused. Moreover, chemical reactions in the solid-state can often be too slow for safe use.

There is a need for alternative methods of detecting the presence of nerve agents in the field that address one or more of the drawbacks of existing techniques. There is a desire for detection methods that have improved sensitivity to detect very low levels of nerve agents. In addition, there is a need for selective detection methods capable of differentiating between different classes of nerve agents.

### SUMMARY OF THE INVENTION

In one aspect, the present invention provides a method for detection of a nerve agent in a sample, which method comprises:
(a) irradiating an optical sensing element, the optical sensing element comprising a fluorescent sensing compound provided on a substrate, and measuring the luminescence of the optical sensing element; wherein the fluorescent sensing compound comprises a combination of at least one electron acceptor moiety and, optionally, one or more electron donor moieties such that the electronic properties of the sensing compound are sufficient to enable a change in the luminescence of the sensing element in the presence of the nerve agent, and a moiety that influences solubility of the sensing compound in a solvent;
(b) contacting the sample with the optical sensing element;
(c) measuring the luminescence of the optical sensing element; and
(d) determining whether the nerve agent is present in the sample based on the measurements obtained in steps (a) and (c).

In preferred embodiments, the fluorescence of the sensing compound is quenched in the presence of the nerve agent.

The optical sensing element may be irradiated continuously or with pulses throughout steps (a), (b) and (c).

It will be understood that the luminescence from the sensing element is effectively continuously measured throughout a method of the invention, therefore steps (b) and (c) are substantially simultaneous. It will be appreciated that a change in luminescence due to quenching will be detected essentially immediately the nerve agent analyte contacts the sensing compound. The degree of quenching may increase with increased contact time. Similarly, quenching will decrease when the analyte separates from the sensing compound.

In an embodiment, in addition to influencing solubility of the sensing compound, the moiety influencing solubility may also influence how the sensing compound interacts with the nerve agent analyte.

In an embodiment, the method is for detection of airborne nerve agents. In an embodiment, the sample is a sample of air, particularly an air sample obtained at a location where the presence of a nerve agent is suspected, such as the scene of a battlefield or terrorist attack.

In an embodiment, the method is selective for V-series nerve agents, for example the method is selective for V-series nerve agents over G-series nerve agents.

In an embodiment, the fluorescent sensing compound is a compound of Formula (I), Formula (Ia), Formula (Ib) or Formula (Ic) as described herein.

In another aspect, there is provided a method as described herein performed in combination with a further detection method. The methods may be carried out sequentially or simultaneously.

The methods of the invention may have application, for example, in battlefield or warfare situations, or in the case of terrorist attack.

### BRIEF DESCRIPTION OF THE DRAWINGS

FIG. 1 is a schematic diagram illustrating a device useful in implementing the detection of nerve agents according to an embodiment of the present invention.

FIG. 2 shows the chemical structure of a selection of sensing compounds (numbered 1 to 8) tested in methods in accordance with the present invention.

FIG. 3 is a graphical representation of photo-induced hole transfer.

FIG. 4 shows a series of graphical representations showing the change in the PL intensity of sensing compounds (compounds 1, 7 and 8) after exposing to a V-series simulant (VO vapour) (˜500 ppb) for 2 min. VO (2 μL) was dropped to the bottom of a measuring optical chamber (volume: 20 mL) and allowed to evaporate for 30 min prior to measurement. The PL of the sensing compound is quenched in the presence of VO. FIG. 4a is a graphical representation of PL intensity of a film of sensing compound 1 to VO vapour (˜500 ppb); FIG. 4b is a graphical representation of PL intensity of a film of sensing compound 7 to VO vapour (˜500 ppb); FIG. 4c is a graphical representation of PL intensity of a film of sensing compound 8 and poly(methyl methacrylate) (PMMA) to VO vapour (˜500 ppb).

FIGS. 5a to 5f shows a series of graphs showing the PL kinetics response for a series of sensing compounds (compounds 1 to 6) in the presence of a V-series simulant (VO vapour). VO (2 μL) was dropped to the bottom of the measuring optical chamber (volume: 20 mL) and allowed to evaporate for 30 min prior to measurement. (i) indicates exposure of a film of sensing compound to VO vapour; (ii) indicates removing the film from the chamber for regeneration. FIG. 5a is a graphical representation of PL kinetics response of a film of sensing compound 1 to VO vapour (˜500 ppb); FIG. 5b is a graphical representation of PL kinetics response of sensing compound 2 to VO vapour (˜500 ppb); FIG. 5c is a graphical representation of PL kinetics response of compound 3 to VO vapour (˜500 ppb); FIG. 5d is a graphical representation PL kinetics response of sensing compound 4 to VO vapour (˜500 ppb); FIG. 5e is a graphical representation of PL kinetics response of sensing compound 5 to VO vapour (˜500 ppb); FIG. 5f is a graphical representation of PL kinetics response of sensing compound 6 to VO vapour (˜500 ppb).

FIG. 6 is a graphical representation of PL kinetics response of a film of sensing compound 1 to diluted VO vapour (˜100 ppb) which was injected three times. Carrier gas N₂ flow rate is 200 mL/min; VO vapour was injected into the carrier gas through a plastic syringe at 50 mL/min.

FIG. 7 is a graphical representation demonstrating the PL kinetics response of sensing compound 1 to VO vapour (a V-series simulant) injected at 5 s without dilution (˜500 ppb). VO vapour was injected into the optical chamber mL in volume) and the injection volume is 0.5 mL, 0.5 mL, 1 mL, 2 mL, respectively, from left to right in the plot.

FIGS. 8a to 8c are graphical representations showing the absence of change in PL intensity of sensing compound 1 before and after exposing to simulant G-series analyte vapour, thus demonstrating that there is no quenching response of sensing compound 1 to simulants of G-series analytes. FIG. 8a is a graphical representation of the PL kinetics response of sensing compound 1 to the saturated vapour of N,N-dimethylformamide (DMF), a simulant of tabun (GA). The arrow indicates when DMF (2 μL) was dropped to the bottom of the optical chamber; FIG. 8b is a graphical representation of the PL kinetics response of sensing compound 1 to the saturated vapour of N,N-dimethyl acetamide (DMAA), a simulant of tabun (GA). The arrow indicates when DMAA (2 μL) was dropped to the bottom of the optical chamber; FIG. 8c is a graphical representation of the PL kinetics response of sensing compound 1 to the saturated vapour of DFP (˜770 ppm), a simulant of sarin (GB), soman (GD), and cyclosarin (GF). The arrow indicates when DFP (2 μL) was dropped to the bottom of the optical chamber. No quenching of the luminescence was observed.

### DETAILED DESCRIPTION OF THE INVENTION

### Abbreviations

As used herein the symbols and conventions used in these processes, schemes, graphs and examples are consistent with those used in the contemporary scientific literature, for example, the Journal of the American Chemical Society. Specifically the following abbreviations may be used in the specification: min (minute); s (seconds); NMR (Nuclear Magnetic Resonance); PL (photoluminescence); CWAs (chemical warfare agents).

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The terms “chemical warfare nerve agent” and “nerve agent” when used herein refer highly toxic synthetic organophosphate compounds classed as chemical weapons. These organophosphate compounds are classed as Schedule 1 poisons. The compounds are usually dispersed in an airborne form, for example as a vapour, a mist or an aerosol. The mode of action is attack of the nervous system though inhibition of acetylcholinesterase in the body, resulting in muscle overstimulation due to buildup of the neurotransmitter acetyl choline. Exposure to nerve agents generally results in death by asphyxiation. Nerve agents are described in, for example, S. Costanzi et al., ACS Chem. Neurosci. 2018, 9, 873; T. C. C. Franca et al., Int. J. Mol. Sci. 2019, 20, 1222; and Mirzayanov, V.S. State Secrets: An Insider's Chronicle of the Russian Chemical Weapons Program; Outskirts Press, Inc.: Parker, CO, USA, 2009; ISBN 1432725661.

Nerve agents are generally divided into two main families, namely the G-series and the V-series. V-series nerve agents have an electron-donating —C—N(alkyl)₂ moiety, i.e., a tertiary amine. The G-series nerve agents do not have a tertiary amine. Where a nitrogen atom is present in a G-series nerve agent, it is generally forms part of a less electron-donating moiety such as —P═O[N(Me)₂]. A third family of nerve agents, the A-series, is commonly referred to as Novichok. These nerve agents are also believed to have a less electron donating —P═O[N═C(NR₂)ₙ] moiety.

Examples of G-series nerve agents include:
Tabun (GA, Ethyl N,N-Dimethylphosphoramidocyanidate);
Sarin (GB, Propan-2-yl methylphosphonofluoridate);
Soman (GD, 3,3-Dimethylbutan-2-yl methylphosphonofluoridate);
Cyclosarin (GF, Cyclohexyl methylphosphonofluoridate).

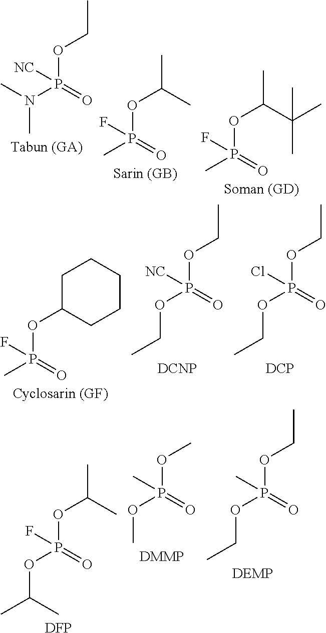

G-series simulants suitable for use in testing the detection methods described herein in a conventional laboratory environment include DCNP (diethyl cyanophosphonate); DCP (diethyl chlorophosphonate); DFP (di-iso-propyl fluorophosphate); DMMP (dimethyl methylphosphonate); DEMP (diethyl methylphosphonate); DMAA (N,N-dimethyl acetamide); and DMF (N,N-dimethylformamide). For example, DFP is a simulant of Sarin, Soman and Cyclosarin. DMAA and DMF are simulants of Tabun.

Examples of V-series nerve agents are well known in the art. Non limiting examples include:
VX [Ethyl ({2-[bis(propan-2-yl)amino]ethyl}sulfanyl)(methyl)phosphinate]; and
R-VX [N,N-diethyl-2-[methyl(2-methylpropoxy)phosphoryl]sulfanyl ethanamine].

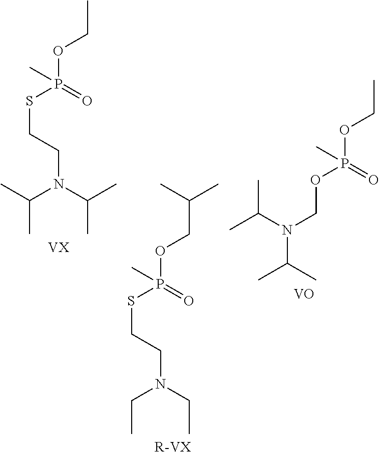

V-series simulants suitable for use in assessing the detection methods described herein in a conventional laboratory environment include VO (2-N,N-di-iso-propylaminoethyl ethyl methylphosphonate).

The chemical structures of the A-series compounds are reported by Mirzayanov (Mirzayanov, V.S. State Secrets: An Insider's Chronicle of the Russian Chemical Weapons Program; Outskirts Press, Inc.: Parker, CO, USA, 2009; ISBN 1432725661). Examples of A-series (Novichok) nerve agents have either a substituted guanidine or imidamide moiety and are reported to include A-230, A-234 and A-262:

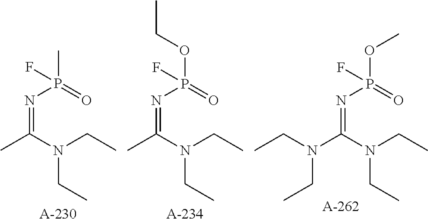

Toxicity data for certain nerve agents is summarized in Table 1. LC₅₀ data (lethal concentration) is obtained from S. W. Wiener, J. Intensive Care Med, 2004, 19, 22-37. LD₅₀ data (lethal dose, g/70 kg man) is derived from T. C. C. Franca et al., Int. J. Mol. Sci. 2019, 20, 1222. LD₅₀ values for A-series nerve agents are estimated [see T. C. C. Franca et al., Int. J. Mol. Sci. 2019, 20, 1222].

TABLE 1
Toxicity Data

| NERVE AGENT | LD₅₀ | LC₅₀ | |
|---|---|---|---|
| Tabun | 1 | 2 | ppm |
| Sarin | 1.7 | 1.2 | ppm |
| Soman | 0.35 | 0.9 | ppm |
| VX | 0.006-0.01 | 0.3 | ppm |

| | | |
|---|---|---|
| A-230 | 0.00075-0.002 | — |
| A-234 | 0.035 | — |
| A-262 | 0.035 | — |

It will be noted that the chemical structures of G-series, V-series and A-series nerve agents and nerve agent simulants may possess at least one chiral (asymmetric) centre. This may be an asymmetric phosphorus atom or asymmetric carbon atom(s). Soman exists as four stereoisomers due to the presence of the asymmetric P atom and the pinacolyl chiral carbon. It will be appreciated that a nerve agent may exist as a single stereoisomer, or as a mixture of isomers, including a racemate. It will also be appreciated that toxicity of different stereoisomers may be different. It will be appreciated that the isomers arising from such asymmetry (e.g., all enantiomers, stereoisomers, diastereomers, rotamers or racemates) are included within the scope of this invention. Where the stereochemistry is not specified, it will be understood that the structure is intended to encompass any stereoisomer and all mixtures thereof.

Chemical weapon nerve agents are often referred to as weapons of mass destruction and, as such, production and stockpiling are restricted under the provisions of the Chemical Weapons Convention (1993). Accordingly, it is not permitted to test nerve agent detection methods under conventional laboratory conditions. For the purposes of research and initial testing, for example in the context of methods described herein, nerve agent simulants may be used to assess applicability of the methods to detection of nerve agents. Nerve agent simulants typically share some structural chemical characteristics with nerve agent molecules allowing them to be detected, but they are less toxic than the nerve agents. Di-iso-propyl fluorophosphate (DFP) is an example of a simulant for G-series nerve agents sarin, soman and cyclosarin. VO is a simulant for the V-series nerve agent, VX.

### Detailed Discussion of the Invention

In accordance with the present invention the presence of a nerve agent can be detected based on the luminescent response of an optical sensing element comprising a fluorescent sensing compound. More specifically, the fluorescent sensing compound can be photoexcited thereby causing a characteristic fluorescent emission. However, when the photoexcited compound is exposed to the nerve agent, quenching of the fluorescence occurs. This quenching can be detected and relied upon as an indicator for the presence of the nerve agent.

It has been discovered that this form of nerve agent detection has particular application for detection of airborne nerve agents, for example in the form of a vapour, mist or aerosol, although it is not necessarily so limited. The detection methods herein may also be applicable to detection of nerve agents in the form of droplets or particles. In one embodiment, the methods described herein are used to detect airborne nerve agents, for example in the form of a vapour.

It will be appreciated that preferably the detection methods described herein operate through the detection of airborne nerve agent, such as a nerve agent in vapour, mist or aerosol form. Known methods for detection of nerve agents generally rely on liquid sampling. The present methods have an advantage of not requiring liquid sampling. Furthermore, the methods described herein can provide a fast response, measured in seconds, thus providing access to detection methods with increased convenience, ease of use, speed and safety.

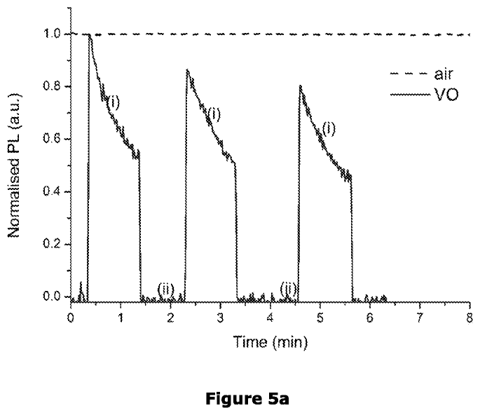

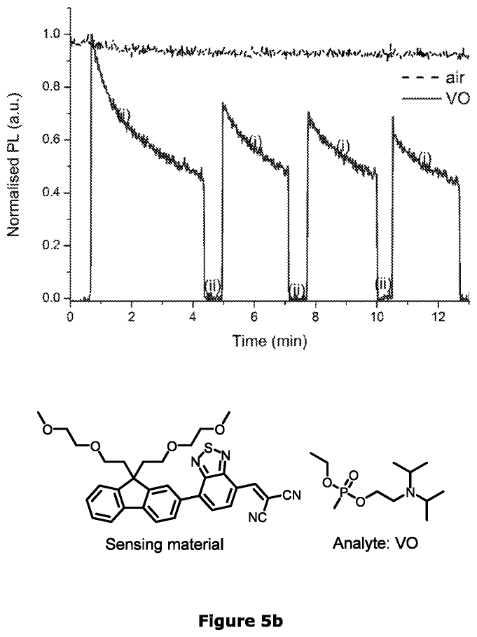

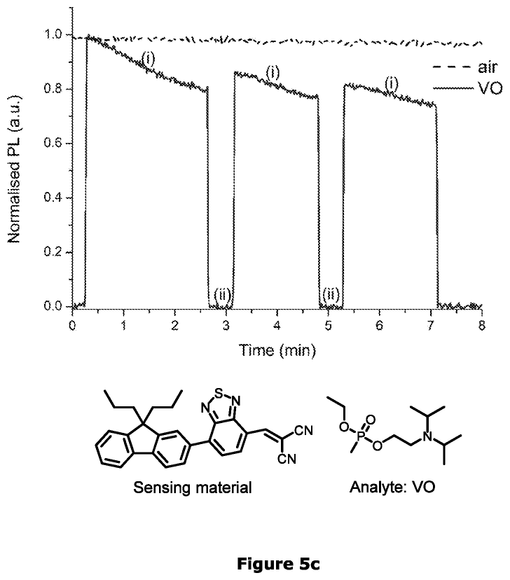

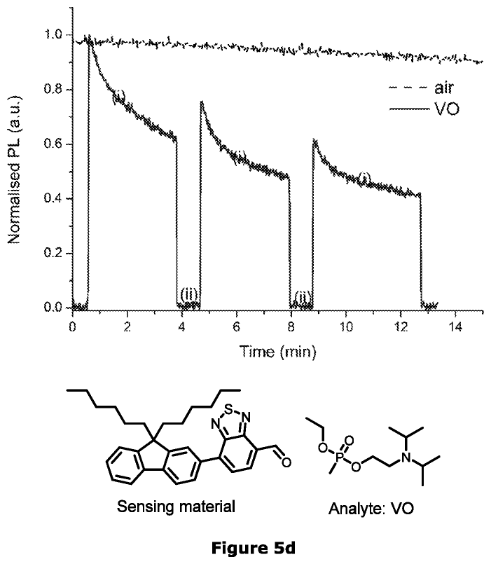

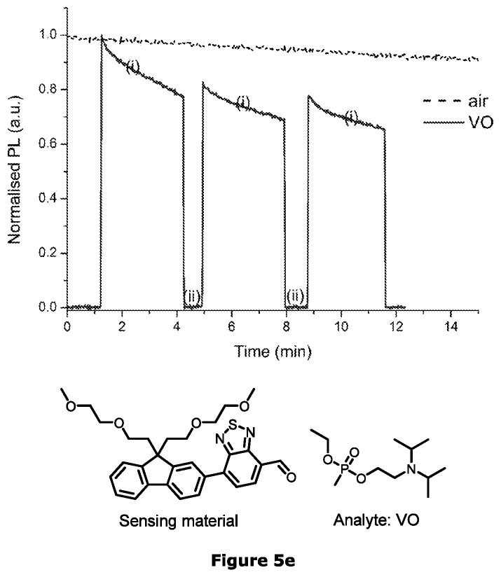

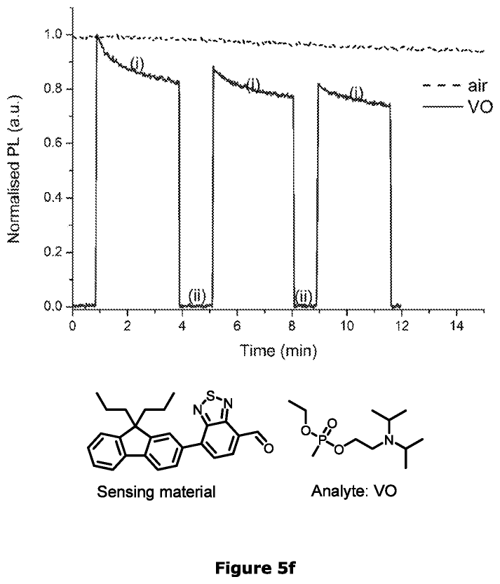

The nerve agent detection methodology described herein is based on charge transfer which is a photoinduced electronic process. Previous methods relying on chemical reaction based transformation cause irreversible chemical changes from one functional group into another on the sensing molecule. The response to the nerve agent analyte in the methods of the current invention can be reversible [see FIGS. 5(a-f), 6 and 7]. This has an advantage over chemical detection methods, a result of this reversibility being that the sensing element can be used multiple times. In comparison, with a chemical reaction-based method, the sensing compound of the sensing element is consumed along with the nerve agent. This limits sensitivity when compared to the present electronic process where the fluorescence of multiple sensing compounds may be quenched by the same molecule of nerve agent analyte, thus providing increased sensitivity and detection of nerve agents at concentrations in the range of parts per billion (ppb). In some embodiments, using the methods described herein, it is possible to detect airborne nerve agents at concentrations down to about 100 ppb. This detection sensitivity is comparable in magnitude to the permissible exposure limits for airborne nerve agents, such as V-series nerve agents.

In some embodiments, the detection methods described herein are believed to be capable of detecting nerve agents in concentrations of less than 500 ppb; less than 400 ppb; less than 300 ppb; less than 250 ppb; less than 200 ppb; or less than 100 ppb, for example down to 50-150 ppb or about 100 ppb.

The detection methods may be used to detect the presence of a V-series nerve agent, for example VX or R-VX. Although not so limited, in some embodiments, the V-series nerve agent is airborne, for example in the form of a vapour, mist or aerosol. In an embodiment, the nerve agent is in the form of a vapour. In an embodiment, the method is selective for V-series nerve agents. In some embodiments, the method is selective for V-series nerve agents over G-series nerve agents.

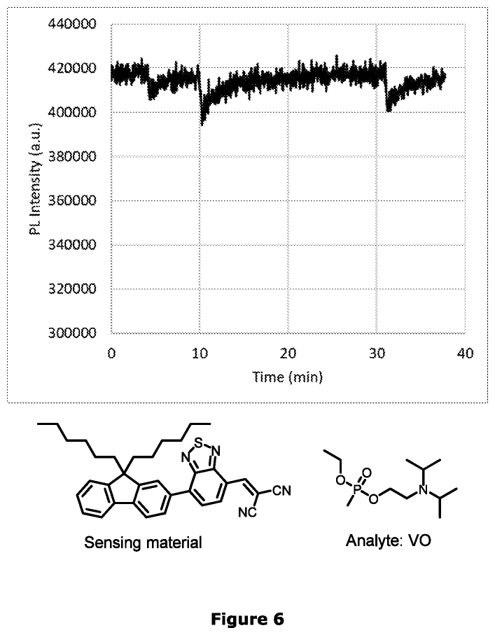

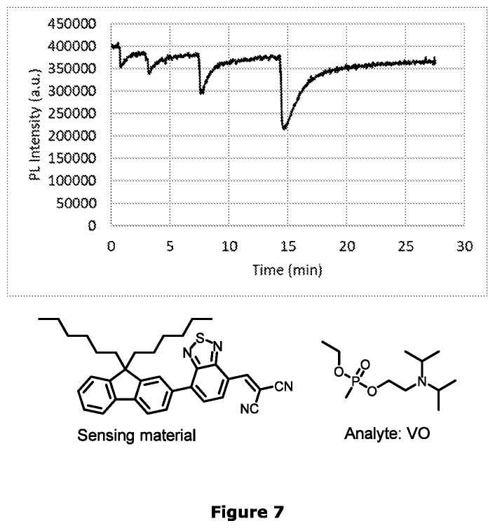

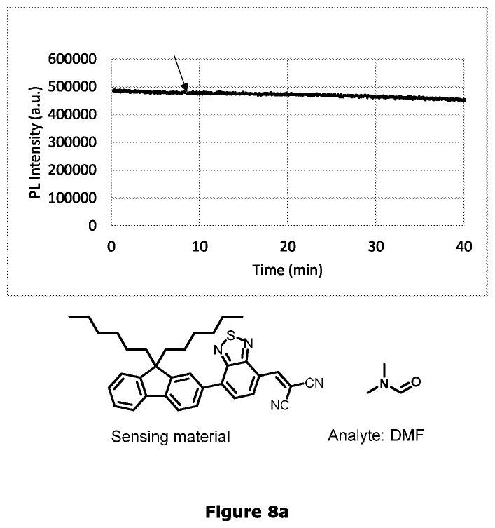

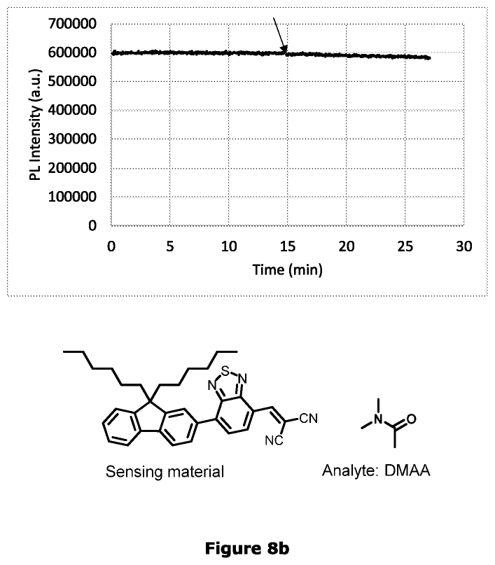

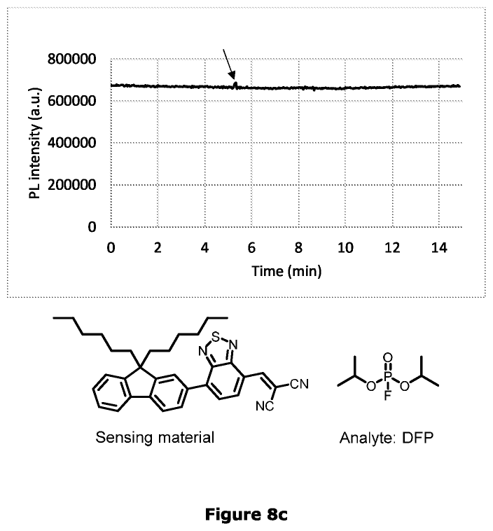

The PL response to VO (a V-series simulant) can be seen in FIGS. 5(a-f), FIG. 6 and FIG. 7, which demonstrates the sensitivity of detection of V-series nerve agents using a method of the invention. There is no PL response to amides (FIGS. 8a and 8b), indicating that the methods will not detect tabun (GA), which has a phosphoramide moiety. There is also no PL response to di-iso-propyl fluorophosphate (DFP) (FIG. 8c), which suggests that these methods cannot be used to detect sarin (GB), soman (GD) or cyclosarin (GF).

Accordingly, the methods described herein may be used to distinguish between V- and G-series nerve agents. This has particular applicability in assessing if a V-series nerve agent, as opposed to a G-series nerve agent, is present in a field situation. The detection method thus provides access to a method of fast detection of a V-series nerve agent to allow appropriate and prompt action to be taken to manage the situation by, for example, adopting appropriate personal protection and countermeasures.

The methods described herein may also provide additional information regarding the identity of the nerve agent. In some aspects, the nerve agent may produce a particular response, such as a characteristic signal shape.

In another aspect, the present invention provides an optical sensing element for detection of a nerve agent, the optical sensing element comprising a fluorescent sensing compound provided on a substrate, wherein the fluorescence of the sensing compound is quenched in the presence of the nerve agent, wherein the fluorescence of the sensing compound comprises a combination of at least one electron acceptor moiety and, optionally, one or more electron donor moieties such that the electronic properties of the sensing material are sufficient to enable a change in the luminescence of the sensing element in the presence of the analyte, and a moiety that influences solubility of the compound in a solvent.

In some embodiments, the sensing compound is non-polymeric and comprises an electron donor moiety, an electron acceptor moiety and a moiety that influences solubility of the compound in a solvent. In some preferred embodiments, the sensing compound is a small molecule.

In some embodiments, the optical sensing element is for detection of airborne nerve agents, for example nerve agents in the form of a vapour, aerosol or mist.

The present invention also provides a sensing device in which the optical sensing element would be used. Accordingly, in this aspect the present invention further provides a sensing device for detection of a nerve agent in a sample, the sensing device comprising:
an optical sensing element as described herein;
an irradiation source for irradiating the optical sensing element with
stimulating radiation;
a detector for measuring luminescence of the optical sensing element; means for delivering the sample for contacting with the optical sensing element; and
means for relating to an operator the luminescence measured by the detector.

In some embodiments, the sensing device is adapted for detection of airborne nerve agents. In some embodiments, the sensing device is for vapour phase detection or adapted for vapour phase detection.

The selective detection of a specific nerve agent analyte may require multiple sensing materials, multiple sensing elements or multiple sensing techniques. The sensing agents used in this invention may be used as key components in a sensor array for selective detection. It will be appreciated that the detection methods described herein may be combined with another technique, such as a colorimetric method or a biosensor to form a binary sensing system.

For example, the compounds of the present invention may be used in conjunction with a colourimetric sensor material that responds to the presence of organophosphates with a colour change. Both sensing materials would therefore respond to the presence of a V-series nerve agent, while only the colourimetric sensor would respond to a G-series nerve agent, thus achieving a greater level of selectivity than with either sensing material on its own.

Fluorescent sensing compounds useful in methods of the present invention require an ionisation potential such that when they are photoexcited the excited sensor molecule can preferably oxidise the V-series nerve agent. In some embodiments, a fluorescent sensing compound has an ionisation potential of at least 5.7 eV; for example greater than 5.8 eV or greater than 6.0 eV. In some embodiments, a fluorescent sensing compound has an ionisation potential of about 5.7 to about 7.5 eV.

The skilled person will appreciate that the combination of chemical moieties in the sensing compound should provide electronic properties in the excited state that are suitable for the required photoinduced hole transfer. It will be understood that a sensing compound for use in the present methods requires at least one electron acceptor moiety. Typically such sensing materials comprise at least one electron acceptor moiety and, optionally, one or more electron donor moieties. Electron donor moieties are defined as having a lower electron affinity and ionisation potential. Electron acceptors moieties are defined as having a higher electron affinity and ionisation potential. These moieties are primarily responsible for providing a chromophore having the correct energetics for the fluorescence quenching of the sensing compound in the presence of the analyte. Thus, a sensing compound may include a chromophore comprising one or more electron acceptor moieties and no electron donor moieties. In some embodiments, a sensing compound may include a chromophore comprising one or more electron acceptor moieties and one or more electron acceptor moieties.

The fluorescent sensing compound also includes one or more further functional moieties that are selected based on their effect on the solubility of the sensing compound in a solvent to permit its deposition on a substrate. In some embodiments, the lipophilicity of the functional moiety can provide an element of selectivity to the nerve agent detected.

In some embodiments, a sensing compound may include more than one chromophore unit wherein the sensing compound does not include chromophore units to the extent that it may be regarded as a polymer. In an embodiment the compound may include units comprising electron donor and electron acceptor moieties with the degree of repetition of those units being up to five. Each repeat unit may comprise one or more electron donor moieties and one or more electron acceptor moieties.

In some embodiments, a plurality of chromophore units form a polymeric sensing compound. Each chromophore unit or monomer (which may be the same or different) comprises one or more electron acceptor moieties and, optionally, one or more electron donor moieties. In some embodiments, the sensing compound is a polymer comprising at least 10 chromophore units. The chromophore units may form the main backbone of the polymer, or may be attached to a polymer chain as a side group, or both. The polymer can be branched or straight chain and can be in the form of a homopolymer or co-polymer. The chomophore units also comprise a moiety that influences solubility. In some embodiments, this solubilising moiety may form at least a portion of the polymer backbone.

The fluorescent sensing compound may include one or more modifier moieties that enable fine tuning of optoelectronic properties of the sensing compound in the context of the present invention. The role of this modifier moiety is to influence how the sensing compound interacts with a nerve agent resulting in a fluorescence quenching effect. The one or more modifier moieties influence the electron affinity of the photoexcited sensing compound with respect to a particular nerve agent. This may enable the selectivity of the sensing compound to be adjusted.

The fluorescent sensing compound may include a branching moiety from which branches comprising the electron donor and electron acceptor moieties extend. In this case it is possible that the branches may include a functional moiety of the type mentioned.

Fluorescent sensing compounds useful in the methods described herein may be represented by formula (I):

(AₐB_{b}C_{c}D_{d}Eₑ)ₘ(L)ₙ (I)
wherein:
A is an electron donor moiety;
B is an electron acceptor moiety;
C is a moiety that influences solubility of the compound in a solvent;
D is a modifier moiety that enables fine tuning of the optoelectronic properties of the sensing compound;
E is a branching moiety;
L is an optional linker group;
a is an integer of 0, 1 or more;
b is an integer of 1 or more;
c is an integer of 0, 1 or more;
d is an integer of 0 to less than or equal to a+b;
e is 0 or 1;
m is an integer of 1 or more;
n is 0 or an integer 1 or more;
wherein:
when m is 1, n is 0 and L is absent; and
when m is an integer greater than 1, preferably greater than 10, L is present and n is an integer of 1 or more thus forming a polymeric sensing compound.

It will be understood that the combination of AₐB_{b}C_{c}D_{d}Eₑ in accordance with the general formula (I) should ensure that the electronic properties of the excited state are suitable for quenching of the fluorescence of the sensing compound by the sample.

It will be appreciated that Formula (I) is an empirical formula and should not be interpreted as implying any particular sequence or arrangement of moieties.

It is also to be understood that definitions given to the variables of the generic formulae described herein will result in molecular structures that are in agreement with standard organic chemistry definitions and atom valencies.

The fluorescent sensing compound may be a dendrimer having a “core” comprising one or more of the electron donor and/or electron acceptor moieties with dendron moieties attached to the core. The dendron moieties may comprise functional moieties of the types mentioned. The dendrons can be first, second or higher generations, with surface groups chosen to provide the necessary solubility and interactions with the analyte.

The sensing compound of Formula (I) may be in the form of a polymer. In this embodiment, the sensing compound includes multiple chromophore units AₐB_{b}C_{c}D_{d}Eₑ within its structure. In this case, m is greater than 1, and preferably greater than 10, and at least one linker group L is present.

When m is greater than 1, it will be understood that at least one linker group L is present in a compound of Formula (I). The linker group (or groups) serve to join the plurality of chromophore units, AₐB_{b}C_{c}D_{d}Eₑ (which may be same or different), to form a polymeric sensing compound of Formula (I). The linker group may be branched or straight chain. It will be understood that the nature of the linker group can vary in function, chemical composition, size and weight depending on the nature of the polymer.

In preferred embodiments, each chromophore is the same. Where more than one linker group is present, each group L may be the same or different.

It will be understood that when the sensing compound of Formula (I) comprises a plurality of chromophore units, the unit could form at least part of the main polymer chain (backbone), or at least part of a side chain, or both. It will also be appreciated that there could be more than one type of chromophore unit in a polymeric sensing compound of Formula (I).

Typical values of m and n will depend on the nature of the polymeric compound of Formula (I), and whether the linker dictates that the chromophore units are linked by L groups to form a polymer backbone, or are attached to a polymer backbone to form a plurality of pendant functional groups or side chains. It will be understood that the polymeric sensing compound may be in the form of, for example, a co-polymer, a homopolymer or a functionalized polymer, for example where the chromophore units are attached to form side chains on a polymer backbone.

In some embodiments of Formula (I), there may be a single linking group L, which may typically be a branched or straight-chain polymer. In this embodiment, typically the plurality of chromophore units (m) will be attached to the polymer chain.

In some embodiments of Formula (I), there may be more than one linking group L, wherein each L moiety serves to link two or more chromophores AₐB_{b}C_{c}D_{d}Eₑ to form a branched or straight-chain polymer.

It will be appreciated that in some cases each chromophore within the polymer will function as an individual chromophore within the polymeric sensing compound. That is, while the individual chromophore may be fully conjugated, the overall structure and arrangement of the polymer is not fully delocalized. This can be achieved by having a linking moiety between the chromophores in the polymer backbone or side chain that does not contain conjugated units.

In some preferred embodiments, the sensing compound is non-polymeric.

Non-polymeric fluorescent sensing compounds of Formula (I) useful in the methods described herein may be represented by formula (Ia):

AₐB_{b}C_{c}D_{d}Eₑ (Ia)
wherein:
A is an electron donor moiety;
B is an electron acceptor moiety;
C is a moiety that influences solubility of the compound in a solvent;
D is a modifier moiety that enables fine tuning of the optoelectronic properties of the sensing compound;
E is a branching moiety;
a is an integer of 0, 1 or more, for example 1 or more;
b is an integer of 1 or more;
c is an integer of 0, 1 or more, for example 1 or more;
d is an integer of 0 to less than or equal to a+b; and
e is 0 or 1.

The compound of Formula (I) may comprise more than one moiety A and more than one B moiety. In this case the formula given should not be interpreted as meaning that the compound necessarily includes a repeat unit —A—B— as other arrangements are possible or contemplated. Other moieties, such as modifier moieties D, can be attached to either A or B, or both. When more than one moiety A is present, they may be the same or different. When more than one moiety B is present, they may be the same or different. When more than one C moiety is present, each C may be the same or different When more than one D moiety is present, each D may be the same or different.

The fluorescent sensing compound may be in the form of a dimer, trimer or higher oligomer. In some embodiments, the fluorescent sensing compound may be a polymer. In an embodiment of formula (I), a is 0. In some embodiments, a is 0 or an integer of 1 to 5. In an embodiment of formula (I), b is an integer of 1 to 5. The moiety A is an electron donor moiety and non-limiting examples of this moiety include fluorenyl, bisfluorenyl, phenyl, thiophenyl, biphenyl, and terphenyl. In some embodiments, A is fluorenyl. The moiety B is an electron acceptor moiety and non-limiting examples of this moiety include benzothiadiazolyl, benzooxadiazolyl, oxazolyl, triazinyl, imidazolyl, pyridinyl and quinoxalinyl. In some embodiments, B is benzothiadiazolyl. In some embodiments, B is pyridinium or naphthalene imide.

Examples of the moiety C include straight or branched chain alkyl or alkoxy groups containing 1 to 10 carbon atoms, for example n-propyl groups; and ethylene glycol chains including 2-methoxymethyl, 2-methoxyethyl, 2-(2-methoxyethoxy)ethyl, and 2-(2-(2-methoxyethoxy)ethoxy)ethyl. The moiety C may include one or more aryl rings (preferably phenyl) and/or hetero atoms, and/or heteroaryl, and/or alkenyl, and/or alkynyl. By way of example the group C may be an alkoxy group, such as C₁₋₁₀ alkoxy attached to a phenyl ring.

Examples of modifier moieties (D) include rhodanine, vinyl cyano esters, vinyl dicyano, vinyl diesters and trifluoroacetyl. For vinyl cyano esters, the ester moiety may include a C₁₋₈ alkyl group. In the case of vinyl diesters, the alkyl groups may be the same or different C₁₋₈ alkyl groups.

In an embodiment, A is fluorenyl and B is benzothiadiazolyl. In an embodiment, A is fluorenyl, B is benzothiadiazolyl and D is 1,1-dicyanovinyl.

The sensing compound may include moieties A and B and, where present, D in a linear arrangement. In this case, e is 0 and no branching moiety is present.

For compounds of Formula (I), in some embodiments, a is 1; b is 1; c is 2; d is 0 or 1; and e is 0. In some embodiments, a is 0; and b is 1.

In an alternative embodiment, the sensing compound may be a branched structure. In this case in Formula (I) or (Ia), e is 1 and the sensing compound includes a branching moiety E to which are attached arms (branches) containing the A, B, C and optionally D and possibly other functional moieties. The number of arms is typically 2, 3, 4, or 6 per molecule of the sensing compound.

In one embodiment the branching moiety is chosen such that each arm behaves as an individual chromophore within the sensing compound. That is, while the sensing compound may be fully conjugated the arrangement of arms is such that the wave functions are not fully delocalized. This can be achieved by having a linking moiety that does not contain conjugated units between the arms, or the use of regio-isomers that break the delocalization such as a meta arrangement of units around a benzene ring, or the use of steric interactions that twist the units out of plane. In an embodiment the branching moiety is a benzene ring substituted by the arms at the 1-, 3- and 5-positions. In another embodiment the branching moiety is a benzene ring itself hexa-substituted by phenylene moieties that form part of the arms. In another embodiment the branching moiety may be an adamantyl moiety tetra-substituted by phenylene moieties which also forms part of the arms.

In a further embodiment the branched material may be a dendrimer comprised of a core, one or more dendrons (branching groups). The use of dendrons may enable the tuning of solubility and intermolecular interactions in the solid state. This may be relevant to controlling analyte diffusion and fluorescence quenching response. The dendrons can be first, second or higher generations, with surface groups chosen to provide the necessary solubility and interactions with the analyte. The dendrimer sensing compounds may have the core comprised of one or more of A and/or B moieties, and the dendrons themselves may contain one or more chromophores comprised of A and/or B and/or other functional moieties. In one embodiment the A moiety or moieties form the first branching point of the one or more dendrons. The dendrons can be comprised of aryl, heteroaryl, vinyl and/or acetylenyl units. For example, the dendron could be comprised of successive layers of 1,3,5-linked phenyl groups. Alternatively, these phenyl units could be linked by one or more (preferably one) vinyl or acetylenyl moieties. The surface groups could be comprised of C moieties.

In some embodiments, the fluorescent sensing compound of Formula (I) or Formula (Ia) is a compound of Formula (Ib):

AₐB_{b}C_{c}D_{d}Eₑ (Ib)
wherein:
A is an electron donor moiety;
B is an electron acceptor moiety;
C is a moiety that influences solubility of the compound in a solvent;
D is a modifier moiety that enables fine tuning of the optoelectronic properties of the sensing compound;
E is a branching moiety;
a is an integer of 1 or more;
b is an integer of 1 or more;
c is an integer of 1 or more;
d is an integer of 0 to less than or equal to b; and
e is 0 or 1.

The compounds of Formula (Ib) are the compounds of Formula (I) as disclosed in WO 2019/0079860 A1 (University of Queensland). The contents of PCT application no. PCT/AU2018/051157, published as WO 2019/0079860 A1 on 2 May 2019, is incorporated herein by reference in its entirety.

For compounds of Formula (I), (Ia) or (Ib), in some embodiments, a is 1; b is 1; c is 2; d is 0 or 1; and e is 0. In some embodiments, d is an integer of 0 to less than or equal to a+b.

In some embodiments, the sensing compounds useful in the invention are small molecules. Typically, a small molecule sensing compound will have a molecular weight of less than 2000, but if the compounds have a dendritic architecture the molecular weight could be higher. Small molecules and dendrimers may offer an advantage of providing reproducibility or consistency over polymeric sensing compounds.

Polymeric sensing compounds are less amenable to reproducible synthesis than small molecules or dendrimers. Typically, batch-to-batch variations during polymer synthesis may result in less reliable or less reproducible sensing properties between batches leading to inconsistent performance between batches.

Some non-limiting examples of fluorescent sensing compounds of Formula (I) that may be useful in the present invention are given below.

Examples of fluorescent small molecule, linear compounds include the compounds identified in WO 2019/079860 A1 (University of Queensland) as: 2.27 (FI-BT); 2.16 (FI-BT-FI); 2.19 (FI-BTBT-FI); 3.4 (K12); 4.8 (K12-Th); 4.20 (K12b); JED; AL03-77; AL03-79; AL03-56; AL03-28; AL03-102; AL03-96; AL04-09; 4.24; 4.7.

Examples of fluorescent branched molecules in which the electron donor and electron acceptor moieties are present in the branches include the compounds identified in WO 2019/079860 A1 as: 2.25 HBP(BT-FI)6; 2.22 Ad(BT-FI)4; K12-3; K12-6; WJ07-24.

Examples of fluorescent dendrimers in which the electron acceptor moieties (B is benzothiadiazolyl and b is 1 or 2) are present in the core of the molecule and electron donor units, (A is phenyl) form the first branching point of a first generation biphenyl dendron with C as 2-ethylhexyloxy include the compounds identified in WO 2019/079860 A1 as 2.10 G1-BT-G1; 2.12 G1-BTBT-G1; WJ05-106; WJ05-113.

In some embodiments, the compound of Formula (I), (Ia) or (Ib) is a fluorescent small molecule linear compound represented by a compound of Formula (Ic):

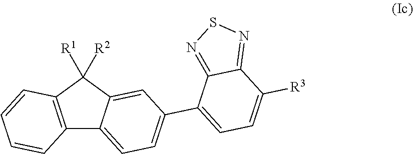

wherein:
R¹ and R² are each independently selected from C₁₋₁₀alkyl, C₁₋₁₀alkoxy or (CH₂CH₂O)ₙC₁₋₄alkyl wherein n is 1, 2 or 3; and
R³ is —CH═C(CN)₂ or —CH═O.

For compounds of Formula (Ic) it will be understood that A is fluorenyl; B is benzothiadiazolyl; C is R¹ and R²; and D is R³; a is 1; b is 1; c is 2; d is 1; and e is 0.

In an embodiment, R¹ and R² are the same. In an embodiment, R¹ and R² are each selected from C₁₋₆alkyl, for example C₃₋₆ alkyl, such as n-propyl (—CH₂CH₂CH₃) or n-hexyl (CH₂(CH₂)₄CH₃). In an embodiment, R¹ and R² are each an ethylene glycol chain selected from —CH₂CH₂OCH₃; —(CH₂CH₂O)₂CH₃ or —(CH₂CH₂O)₃CH₃. In an embodiment, R³ is —CH═C(CN)₂.

In an embodiment, the compound of Formula (I), (Ia), (Ib) or (Ic) is selected from:

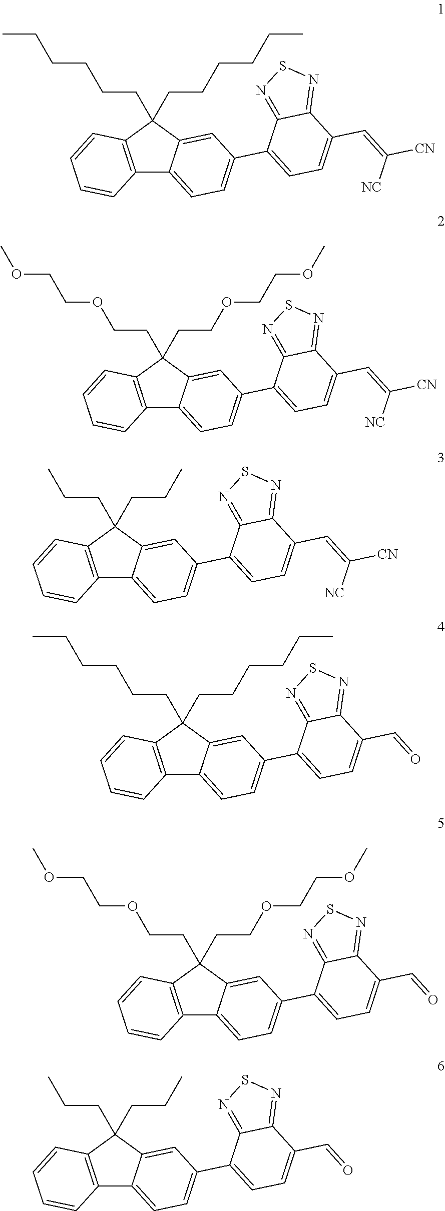

In some embodiments, the sensing compound is Compound 1, Compound 2 or Compound 3.

In some embodiments, the sensing compound is a perylene diimide, a naphthalene imide, or a pyridinium compound. In some embodiments, the sensing compound is not a perylene diimide. In some embodiments, the compound of Formula (I) or (Ia) is a naphthalene imide compound wherein a is 0, b is 1 and the electron acceptor moiety B is a naphthalene imide, for example Compound 7:

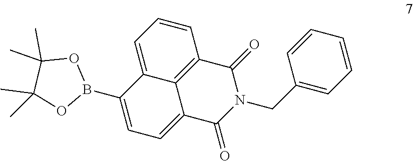 [SMILES: CC1(C)OB(c2ccc3c4c(cccc24)C(=O)N(Cc2ccccc2)C3=O)OC1(C)C]

In some embodiments, the compound of Formula (I) or (Ia) is a pyridinium compound wherein a is 0, b is 1 and the electron acceptor moiety B is pyridinium, for example Compound 8:

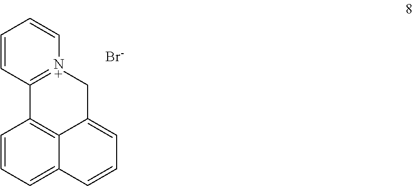 [SMILES: c1cc[n+]2c(c1)-c1cccc3cccc(c13)C2 ; [Br-]]

In some embodiments, a sensing compound as described herein is novel. Accordingly, the present invention also encompasses novel sensing compounds as described herein, for example Compound 7 and Compound 8 as defined above.

Compounds of Formula (I), (Ia), (Ib) or (Ic) may be synthesized using recognized multi-step synthetic routes known in the art. Non-limiting examples of fluorescent sensing compounds useful in the methods described herein are described in, for example, Ke Gui, PhD Thesis entitled “Novel materials for bulk heterojunction thin film organic photovoltaic devices—research and application”, The University of Queensland, Australia, 2012, which is viewable in UQ eSpace (https://doi.org/10.14264/uql.2017.795); J. E. Donaghey, A. Armin, Dani M. Stoltzfus, P. L. Burn, P. Meredith, “Dielectric constant enhancement of non-fullerene acceptors via side-chain modification”, Chem. Comm., DOI: 10.1039/c6cc90512a, WO 2019/079860 A1 (University of Queensland) or in accordance with the synthetic routes described in the examples below.

Examples of polymeric fluorescent sensing compounds are described in, for example, Y. Fu et al., Polym. Chem., 2015, 6, 2179-2182 and S. Rochat and T. M. Swager, Angew. Chem. Int. Ed., 2014, 53, 9792-9796.

In use in accordance with a method of the present invention, a photoexcited sensing compound interacts with a nerve agent thereby causing a fluorescence quenching effect. This interaction can take place at ambient temperature and pressure, which makes it particularly useful in the field and simplifies design of a sensing device in which the sensing compound/optical sensing element is used. However, this is not essential and it is possible that the interaction takes place at elevated temperature and the device designed accordingly to facilitate this. For example, the device may require some form of heating means to raise the temperature of the optical sensing element to provide a stable testing temperature independent of the environment or change the absorption/desorption kinetics.

The mechanism that causes fluorescence quenching of the photoexcited sensing compound when exposed to the nerve agent is photo-induced hole transfer (PHT). That is, on excitation of the sensing compound an electron is transferred from the analyte to the sensing molecule leading to a pathway by which the excited state can then undergo non-radiative decay. For this to take place the energy of the highest occupied molecular orbital (HOMO) or ionisation potential of the sensing compound must be such that, on excitation, the exciton can oxidise the nerve agent. The fluorescence quenching effect when the nerve agent interacts with photoexcited sensing compound may be transient. The reaction may thus be reversible and the optical sensing element may therefore be reusable.

In an embodiment of the optical sensing element, the sensing compound is provided as a thin film coating on a solid transparent substrate. The term “transparent” refers to the ability of the substrate to allow transmission of electromagnetic radiation used for photoexcitation of the sensing compound. In this embodiment the optical sensing element is therefore a solid-state system. The sensing compound will typically be provided as a continuous layer (coating) on the transparent substrate. To produce the coating the sensing compound may be dissolved in a solvent and applied to the substrate using conventional means for coating. The solvent is then removed leaving the sensing compound as a coating on the substrate. Examples of suitable solvents that may be useful in the practice of the invention include toluene; chlorinated solvents such as dichloromethane and chloroform; acetone; ethanol; methanol; iso-propanol; tert-butanol; methoxyethanol; tetrahydrofuran (THF); N,N-dimethylformamide (DMF); dimethyl sulfoxide (DMSO); 1,3-dioxane and 1,4-dioxane, or a mixture thereof.

Typically the film coating will have a thickness of 100 nm, or less. In some embodiments, the film coating has a thickness of 10 nm to 100 nm or 50 nm to 100 nm, for example 50 nm to 80 nm. In some embodiments, the coating is a thin coating of 20 nm to 50 nm, for example 20 nm to 30 nm, or 25 nm to 35 nm.

In the optical sensing element, a polymer may also be employed with the sensing compound to form the coating. This may be appropriate in scenarios that require large-area and/or thick coatings or to change the polarity of the film. Examples of suitable polymers that may be useful in practice of the invention include polyethylene oxide (PEO), poly(methyl methacrylate) (PMMA) and cellulose acetate. Methods of coating a substrate are well known, and will depend on the shape, configuration and/or chemical composition of the substrate.

The minimum amount of sensing compound provided in the coating will be that required to produce a detectable fluorescent emission when excited and a detectable fluorescence quenching when the photoexcited sensing compound is exposed to a nerve agent. The amount of sensing compound included in the coating may be determined experimentally. Determination of the amount of sensing compound will be well within the skill and knowledge of the person skilled in the art. The thickness of the sensing film will determine how quickly signal saturation is reached, with thicker films taking longer and potentially allowing for multiple detection events before any recovery. When the response from the interaction with the nerve agent is reversible the films can be reused and the thickness only needs to be such that a measurable change in the fluorescence is observable in each sensing event.

The substrate may take a variety of forms, and may depend on the phase employed in the method of detection. Methods of detection include those where the analyte is airborne and in the form of a vapour, aerosol or mist. In some methods, the analyte is in solution. It will be appreciated that the nature of the substrate should be compatible with the detection technique with regard to, for example, resistance to solubility, chemical compatibility with sensing compound and/or degradation by heat, light or chemical reaction. For example, a substrate may be a glass such as borosilicate glass or fused silica.

It will be appreciated that methods for detection of airborne analytes will be of particular use in the field of detection of nerve agents, for example on a battlefield or other warfare situation, or at the scene of a terrorist attack, suspected terrorist attack or laboratory accident. For airborne analyte detection, such as vapour phase detection, the substrate may take the form of, for example, a tube or a surface in an enclosed channel with the sensing compound provided as a coating on an internal surface of the tube or surface in the channel. In this case a sample to be tested is provided to the interior of the tube or enclosed channel for contacting where it will come into contact with the sensing compound. Where the substrate is a tube it may be a capillary tube made of a glass, such as a borosilicate glass or silica. Typically the capillary tube will have an internal diameter of up to 1 mm. The length of the capillary tube is usually no more than 100 mm. Capillary tubes useful in the invention are commercially available and may be cut to an appropriate length.

A desirable property of the optical sensing element of the invention is that it is non-scattering when irradiated, as takes place during the detection process. Preferred substrates are transparent. However, in certain applications and configurations reflective substrates may also be useful.

In some embodiments, the response from the interaction of the sensing compound with the nerve agent is reversible. Reversibility typically occurs when the sensing element recovers its original detection properties after the analyte is removed. In such circumstances, the sensing element may be used again. It will be appreciated that an activator may be required to promote reversibility. Reversibility may be encouraged by heating the sensing element.

The present invention uses an irradiation source for irradiating the optical sensing element with stimulating radiation in order to photoexcite the sensing compound prior to contacting with a sample that may include a nerve agent to be detected. Generally the exciting radiation is in the UV or near UV-deep blue or blue register.

A detector is used for measuring any luminescent response (quenching) of the optical sensing element when photoexcited and after exposure to a sample. It is envisaged that the luminescent response will be measured with a broadband detector such as a photodiode. To maximize sensitivity an amplified detector such as an avalanche photodiode or photomultiplier tube could be used. Alternatively, a spectrally resolved detector such as CCD spectrograph may be used to resolve changes in the luminescence shape and intensity. In addition, a long-pass or band-pass optical filter may be included between the sensor and the detector to block the excitation wavelength from reaching the detector. The detection will include some means for relating to an operator the luminescence measured by the detector. This may involve some form of signal, for example a signal that is communicated visually, audibly or stimulatorily (for example by vibration).

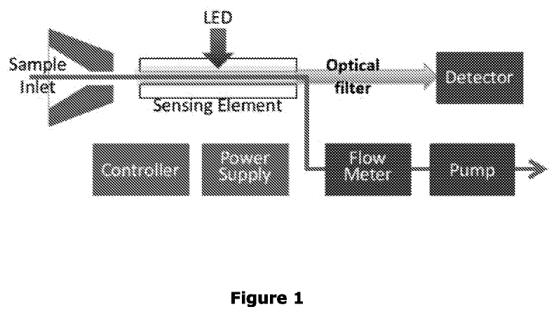

The device of the invention will also include a means for delivering a sample to be analysed for contacting with the (photoexcited) optical sensing element. FIG. 1 depicts components that may be present in a device useful for implementing the present invention.

For detection of airborne analyte, the sample will be in a form such as a vapour, aerosol or mist. Typically, this means a fan or blower or pump coupled with a flow meter will be needed to continuously draw the sample into contact with the optical sensing element. For solution phase detection, the sample will be in solution and typically the sample may be drawn into contact with the sensing element using a pump.

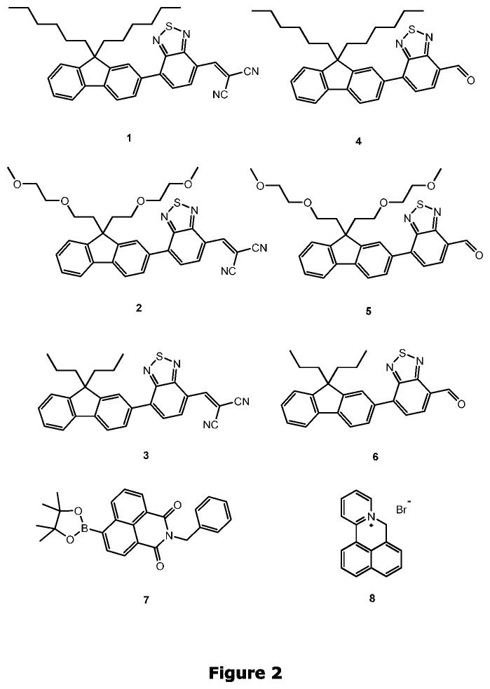

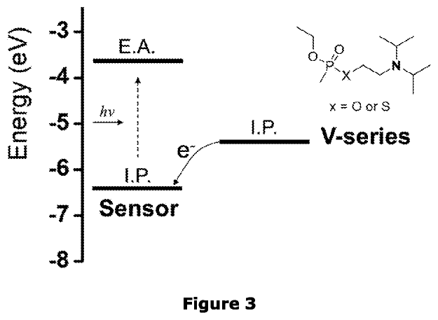

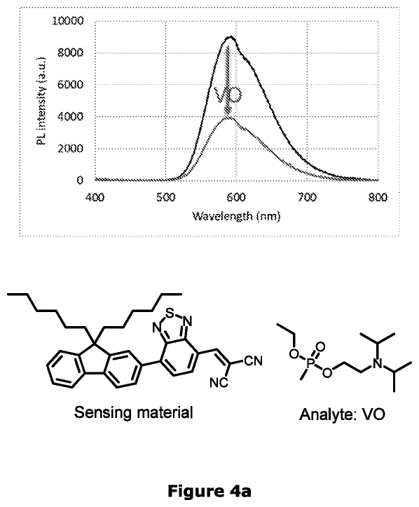

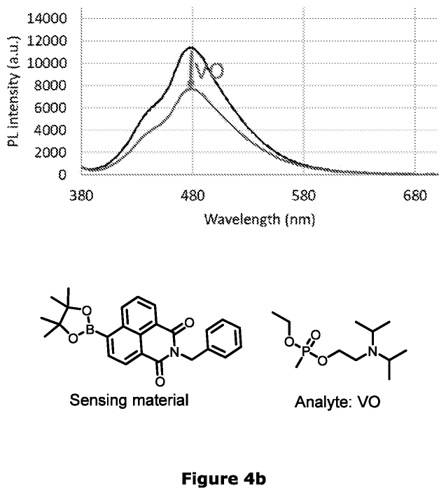

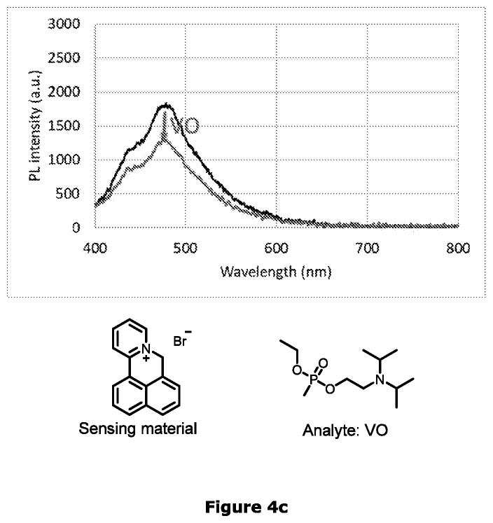

As can be seen from the molecular structures of V-series nerve agents, these compounds possess an electron-donating functionality in the form of a tertiary amine. This feature can be used to distinguish between V- and G-series nerve agents. Although tabun (GA) and the Novichok A-series nerve agents also have a tri-substituted nitrogen atom, it is attached to electrophilic —P═O and —C═N moieties, respectively. Thus, it will be understood that the electron-donating ability is significantly decreased. The mechanism to detect V-series nerve agents is photo-induced charge transfer. This is summarized graphically in FIG. 3. FIG. 2 shows a selection of fluorescent sensing materials useful in the methods of the invention. When they are photo-excited, electron transfer occurs from the amino functionality to the fluorescent sensing material and this leads to quenching of the fluorescence signal (FIG. 4).

It will be appreciated that the methods described herein may be combined with an additional method to reinforce evidence for the presence of a nerve agent. Examples of additional detection methods for use in combination with the methods described herein include commercially available colorimetric detection paper [V. Pitschmann, et at,, Chemosensors 2019, 7, 30] or a commercially available biosensor [L. Matějovský, V. Pitschmann, Biosensors 2018, 8, 51]. An additional detection method which may be used in combination with the sensing material described herein is a secondary fluorescent sensing material which can specifically detect organophosphonate/organophosphate functionality.

It will be understood that when a detection method as described herein is used in combination with another detection method, this combined methodology will serve to reinforce or confirm the presence of a nerve agent. It will be appreciated that detection methods may be used sequentially or simultaneously, and in any order. In an embodiment, a combined method may be carried out simultaneously using an array of sensors.

In order that the invention may be readily understood and put into practical effect, particular preferred embodiments will now be described by way of the following non-limiting examples.

### EXAMPLES

Unless otherwise noted, all starting materials, solvents and reagents were obtained from commercial suppliers and used without further purification.

Nuclear Magnetic Resonance Spectra was performed at 400 or 500 MHz (¹H) or 121.5 MHz (³¹P). ¹H and ³¹P chemical shifts are given in parts per million (ppm) using residual protonated solvent (CD₂Cl₂, CDCl₃, or CD₃OD) as an internal standard. Coupling constants (J) are quoted in Hertz (Hz). The following abbreviations are used: s (singlet), d (doublet), t (triplet), q (quartet), m (multiplet), br (broad).

Chemical warfare nerve agent simulants such as DCNP, DCP, DFP, DMMP, DMMP, DMF and DEMP are available from commercial sources. It will be understood that DCNP, DCP and DFP may hydrolyse to produce acidic impurities. Hydrolysis may occur on storage, therefore some commercial sources of DFP are found to contain acids. [D. R. Heiss et al., J. Chem. 2016, 3190891]. An acid scavenger such as poly(4-vinylpyridine) (PVP), hexamethylenetetramine (HMTA), or piperidinomethyl polystyrene (PDN-PS) can be added to the analyte to remove acid impurities before the sensing measurement [K. J. Wallace et al., Chem. Commun. 2006, 45, 3886. R. Zhu et al., Angew. Chem. Int. Ed. 2016, 55, 9662.] With the sensing compound 1, there is no response to hexamethylenetetramine (HMTA) treated DFP (FIG. 8c). There is also no response to non-treated DFP which may contain acid impurities. With an acid analyte, acetic acid, no response is observed. This indicates that these sensing compounds may have no response to both pure and impure G-series, Sarin, Soman and Cyclosarin.

### Example 1: Synthesis of VX/R-VX Simulant (VO)

2-N,N-Disopropylaminoethyl ethyl methylphosphonate (VO) was synthesised following a reported method [G. H. Dennison et al., Supramolecular Agent-Simulant Correlations for the Luminescence Based Detection of V-Series Chemical Warfare Agents with Trivalent Lanthanide Complexes, Eur. J. Inorg. Chem. 2016, 1348-1358. doi:10.1002/ejic.201600105] using ethyl methylphosphonochloridate and 2-diisopropylaminoethanol in the presence of 4-(N,N-dimethylamino)pyridine in acetonitrile. ¹H NMR (300 MHz, CD₂Cl₂): δ1.00 (d, J=5.5 Hz, 12 H, 4×NCH₂CH₃), 1.30 (t, J=7.0 Hz, 3 H, OCH₂CH₃), 1.42 (d, J=17.5 Hz, 3 H, PCH₃), 2.63 (t, J=7.5 Hz, 2 H, NCH₂), 3.00 (sept, J=6.5 Hz, 2 H, 2×CH), 3.75-3.93 (m, 2 H, OCH₂CH₂N), 3.98-4.11 (m, 2 H, OCH₂CH₃), ³¹P NMR (121.5 MHz, CD₂Cl₂): δ=30.1 ppm. Purity 97%, ³¹P NMR spectroscopy.

### Example 2: Synthesis of Sensing Compound 7

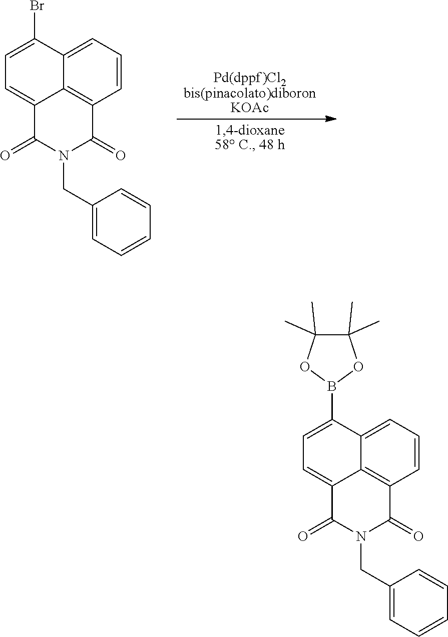 [SMILES: O=C1c2cccc3c(Br)ccc(c23)C(=O)N1Cc1ccccc1 ; "1,4-dioxane58° C., 48 h" ; "Pd(dppf)Cl2bis(pinacolato)diboronKOAc" ; CC1(C)OB(c2ccc3c4c(cccc24)C(=O)N(Cc2ccccc2)C3=O)OC1(C)C]

A mixture of 2-benzyl-6-bromo-1H-benzo[de]isoquinoline-1,3(2H)-dione [K. A. MacGregor et al., J. Med. Chem., 2014, 57 (1) 131-143.] (550 mg, 1.50 mmol), bis(pinacolato)diboron (760 mg, 1.80 mmol), potassium acetate (890 g, 5.40 mmol), 1,1′-bis(diphenylphosphino)ferrocene-palladium(II)-dichloride dichloromethane complex (110 mg, 0.15 mmol) and anhydrous 1,4-dioxane (30 mL) was stirred at 58° C. under argon in an oil bath for 48 h. After being allowed to cool to room temperature, dichloromethane (50 mL) was added and the mixture was filtered to remove the insoluble solid. The filtrate was collected and the solvent removed. The residue was purified by column chromatography over silica gel using ethylacetate:petroleum ether (1:4) as eluent to afford the product as a white solid (389 mg, 63%). mp 165-166° C. Elemental analysis (%) calcd for C₂₅H₂₄BNO₄ C 72.7, H 5.85, N 3.4; Found: C 72.3, H 5.9, N 3.35. λₘₐₓ(dichloromethane)/nm: 301 sh (logε/dm³ mol⁻¹ cm⁻¹ 3.42), 329 sh (3.91), 342 (4.04), 358 (4.00). λₘₐₓ(em) (dichloromethane)/nm: 371, 390, 410 sh, 437 sh. ¹H NMR (δ, 400 MHz, CDCl₃): 1.45 (12 H, s, —CH₃), 5.38 (2 H, s, —CH₂—), 7.21-7.26 (1 H, m, Phenyl-H), 7.28-7.33 (2 H, m, Phenyl-H), 7.54-7.57 (2 H, m, Phenyl-H), 7.75 (1 H, t, J=8.0, Naphthalenyl-H), 8.28 (1 H, d, J=7.0, Naphthalenyl-H), 8.57 (1 H, d, J=7.0, Naphthalenyl-H), 8.60 (1 H, d, J=7.0, Naphthalenyl-H), 9.11 (1 H, dd, J=1.0, J=8.5, Naphthalenyl-H). ¹³C NMR(δ, 100 MHz, CDCl₃): 24.9, 43.6, 84.6, 122.5, 124.6, 127.0, 127.4, 127.8, 128.4, 128.9, 129.9, 131.0, 135.1, 135.2, 135.7, 137.3, 164.3. MS (ESI⁺): m/z expected for [M+Na]⁺436.2, found: 436.0.

### Example 3: Synthesis of Sensing Compound 8

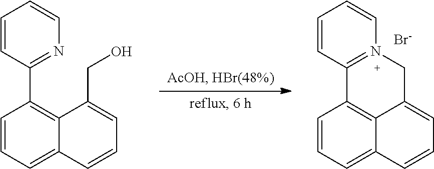

A mixture of (8-(pyridin-2-yl)naphthalen-1-yl)methanol [S.-W. Zhang, T. M. Swager, J. Am. Chem. Soc. 2003, 125, 3420.] (130 mg, 0.53 mmol), glacial acetic acid (8 mL) and hydrobromic acid (48%, 8 mL) was stirred at reflux under argon in an oil bath for 6 h. After being allowed to cool to room temperature, water (100 mL) was added and the mixture was extracted with dichloromethane (4×50 mL) and a mixture of dichloromethane:methanol (4:1) (2×50 mL). The organic extracts were combined, dried over anhydrous sodium sulfate, and filtered. The filtrate was collected and the solvent removed. The residue was purified by recrystallization with methanol/diethyl ether three times to give Compound 8 as a yellow solid (93 mg, 59%). mp 229-230° C. Elemental analysis (%) calcd for C₁₄H₁₁NBr C 64.45, H 4.1, N 4.7; Found: C 64.1, H 4.1, N 4.4. λₘₐₓ(ethanol)/nm: 276 (logε/dm³ mol⁻¹ cm ⁻¹ 3.95), 327 sh (3.48), 346 sh (3.62), 376 (3.86). λₘₐₓ(fluorescence) (ethanol)/nm: 479. ¹H NMR (δ, 500 MHz, MeOD): 6.28 (2 H, s, Methylene-H), 7.68 (1 H, ddt, J=1.5, J=1.5, J=7.0, Naphthalenyl-H), 7.72 (1 H, t, J=7.0, Naphthalenyl-H), 7.79 (1 H, t, J=8.0, Naphthalenyl-H), 7.98 (1 H, ddd, J=1.5, J=6.0, J=7.5, Pyridyl-H), 8.01 (1 H, dd, J=1.0, J=8.0, Naphthalenyl-H), 8.25 (1 H, d, J=8.5, Naphthalenyl-H), 8.58 (1 H, ddd, J=1.5, J=7.0, J=8.5, Pyridyl-H), 8.62 (1 H, d, J=7.5, Naphthalenyl-H), 8.89 (1 H, ddd, J=1.0, J=1.0, J=6.5, Pyridyl-H), 8.93 (1 H, d, J=8.0, Pyridyl-H). ¹³C NMR (δ, 125 MHz, MeOD): 58.2, 123.1, 125.3(8), 125.3(9), 126.2, 126.3, 126.8, 127.1, 128.1, 128.8(1), 128.8(3), 134.2, 135.3, 146.2, 146.6, 150.3. m/z (HRMS-ESI⁺): expected for [M-Br]⁺218.0964, found 218.0963.

### Example 4: Preparation of Solid-State Sensing Films

Film preparation: The sensing compound was dissolved in chloroform (distilled from potassium carbonate, 10 mg/mL) and thin films were prepared on fused silica substrates by spin coating. The thickness is ˜20 nm (Specialty Coating Systems, G3P-8, 5000 RPM, 60 sec dwell, 1 sec ramp) to produce thin films of the sensing compound on the substrate.

The sensing compound was also mixed with a polymer such as PMMA (weight ratio 1:20) and dissolved in chloroform and thin films were prepared on fused silica substrates by spin coating. The thickness is ˜220 nm (Specialty Coating Systems, G3P-8, 2000 RPM, 60 sec dwell, 1 sec ramp) to produce thin films of the sensing compound and the polymer on the substrate.

### Example 5: Generation of Analyte Vapour

Headspace vapour. 2 μL of analyte was added on the surface of a Teflon® lid which was placed at the bottom of the sample chamber. Evaporation under ambient conditions (approximately 22° C.) provided analyte vapour.

Diluted vapour. An analyte, VO, was diluted using two methods for sensing measurement. (i) VO vapour was injected to nitrogen carrier gas (flow rate is 200 mL/min) through a plastic syringe at 50 mL/min and the estimated VO vapour is ˜100 ppb; (ii) A certain volume (0.5, 1, 2 mL) of saturated VO vapour was injected to the optical chamber.

### Example 6: Sensing Measurement

The sensing film samples on fused silica substrates were mounted in a closed sample chamber which was connected to an LED light source (365 nm, OceanOptics) and a spectrometer (Flame, OceanOptics). The sample chamber possessed three optical windows to allow for excitation of the films and subsequent detection of the film PL at right angles to the excitation. Film PL spectra before and after exposure to analyte and PL kinetics at the emissive peak were recorded through OceanView software (OceanOptics).

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A method for detection of a nerve agent in a sample, which method comprises:
(a) irradiating an optical sensing element, the optical sensing element comprising a fluorescent sensing compound provided on a substrate, and measuring the luminescence of the optical sensing element; wherein the fluorescent sensing compound comprises a combination of at least one electron acceptor moiety and, optionally, one or more electron donor moieties such that the electronic properties of the sensing compound are sufficient to enable a change in the luminescence of the sensing element in the presence of the nerve agent, and a moiety that influences solubility of the sensing compound in a solvent;
(b) contacting the sample with the optical sensing element;
(c) measuring the luminescence of the optical sensing element; and
(d) determining whether the nerve agent is present in the sample based on the measurements obtained in steps (a) and (c).

2. A method according to claim 1, wherein the method is for detection of airborne nerve agent, particularly vapour phase.

3. A method according to claim 2, wherein the method is selective for a V-series nerve agent.

4. A method according to claim 3, wherein the nerve agent is VX or R-VX.

5. A method according to claim 1, wherein the fluorescent sensing compound is provided as a thin film coating.

6. A method according to claim 5, wherein the fluorescent sensing compound is a compound of formula (I):
(AₐB_{b}C_{c}D_{d}Eₑ)ₘ(L)ₙ (I)
wherein:
A is an electron donor moiety;
B is an electron acceptor moiety;
C is a moiety that influences solubility of the compound in a solvent; D is a modifier moiety that enables fine tuning of the optoelectronic properties of the sensing compound;
E is a branching moiety;
L is an optional linker group;
a is an integer of 0, 1 or more;
b is an integer of 1 or more;
c is an integer of 0, 1 or more;
d is an integer of 0 to less than or equal to a+b;
e is 0 or 1;
m is an integer of 1 or more;
n is 0 or an integer 1 or more;
wherein:
when m is 1, n is 0 and L is absent; and
when m is an integer greater than 1, L is present and n is an integer of 1 or more.

7. A method according to claim 6, wherein the fluorescent sensing compound is a compound of Formula (Ia):
AₐB_{b}C_{c}D_{d}Eₑ) (Ia)
wherein:
A is an electron donor moiety;
B is an electron acceptor moiety;
C is a moiety that influences solubility of the compound in a solvent;
D is a modifier moiety that enables fine tuning of the optoelectronic properties of the sensing compound;
E is a branching moiety;
a is an integer of 0, 1 or more, for example 1 or more;
b is an integer of 1 or more;
c is an integer of 0, 1 or more, for example 1 or more;
d is an integer of 0 to less than or equal to a+b; and e is 0 or 1.

8. A method according to claim 6, wherein the fluorescent sensing compound is a compound of Formula (Ic): 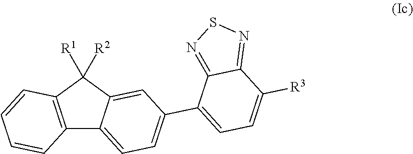
wherein:
R¹ and R² are each independently selected from C₁₋₁₀alkyl, C₁₋₁₀alkoxy or (CH₂CH₂O)ₙC₁₋₄alkyl, wherein n is 1, 2 or 3; and
R³ is —CH═C(CN)₂ or —CH═O.

9. A method according to claim 1, performed in combination with a further detection method.

10. A method according to claim 9, wherein the methods are performed simultaneously or sequentially.

11. A method according to claim 10, wherein the further detection method is a colorimetric detection method.
